(19) United States
(12) Plant Patent Application Publication
(10) Pub. No.: US 2007/0283470 P1
(43) Pub. Date: Dec. 6, 2007

(54) Lomandra longifolia plant named 'LMV100'

(76) Inventor: Todd Anthony Layt, Richmond (AU)

Correspondence Address: Versa Scapes, LLC, P.O. Box 2108, Mount Pleasant, SC, 29465, US

(21) Appl. No.: 11/810,224

(22) Filed: Jun. 5, 2007

Related U.S. Application Data
(60) Provisional application No. 60/810,893, filed on Jun. 5, 2006.

(51) Int. Cl. A01H 5/00

(52) U.S. Cl. PLT/263

(57) ABSTRACT

‘LMV100’ is a distinctive variety of Lomandra longifolia which is characterized by the combination of its variegated leaves, medium density plant growth habit, medium-tall plant height and upright to semi-upright growth habit.

### LATIN NAME OF THE GENUS AND SPECIES

The Latin name of the genus and species of the novel variety disclosed herein is Lomandra longifolia.

### VARIETY DENOMINATION

The inventive variety of Lomandra disclosed herein has been given the variety denomination ‘LMV100’.

### BACKGROUND OF THE INVENTION

The present invention relates to a new and distinct perennial variety of Lomandra longifolia, which has been given the variety denomination of ‘LMV100’. Its market class is that of an ornamental grass-like plant. ‘LMV100’ is intended for use in landscaping and as a decorative grass-like plant.

The Lomandra longifolia variety ‘LMV100’ was first discovered in 2000 in an Australian nursery in the state of New South Wales during an inspection of large quantities of Lomandra longifolia seedlings from open pollinated ‘Cassica’ (unpatented) production stock. ‘LMV100’ is a seedling selection of open pollinated Lomandra longifolia ‘Cassica’ (unpatented). Possible pollinators include Lomandra longifolia ‘Cassica’ (unpatented) and Lomandra longifolia ‘Katrinus’ (unpatented), which was growing nearby. ‘LMV100’ was selected due to its variegated leaf. ‘LMV100’ was first propagated asexually by division in the state of New South Wales, Australia and has since been asexually propagated by division and micropropagation. The distinctive characteristics of the inventive ‘LMV100’ variety are stable from generation to generation; clones of the variety produced by asexual reproduction maintain the distinguishing characteristics of the original plant.

‘LMV100’ has a medium density growth habit with leaves that are variegated whereas Lomandra longifolia ‘Katrinus’ (unpatented in the United States; Australian Plant Breeders' Rights Application No. 1997/168), ‘Cassica’ (unpatented in the United States; Australian Plant Breeders' Rights Application No. 1997/166) and common Lomandra longifolia are non-variegated.

An application for plant breeders' rights for variety ‘LMV100’ has been lodged with the Australian Plant Breeders' Rights Office, and was accepted on Jun. 29, 2005 under Application No. 2005/180.

### SUMMARY OF THE INVENTION

‘LMV100’ is a distinctive variety of Lomandra longifolia which is characterized by the combination of its variegated leaves, medium density plant growth habit, medium-tall plant height and upright to semi-upright growth habit.

### BRIEF DESCRIPTION OF THE FIGURES

FIG. 1 shows an ‘LMV100’ plant showing growth habit.

FIG. 2 shows an ‘LMV100’ plant base detailing color of basal leaf sheath.

FIG. 3 shows an ‘LMV100’ leaf sections showing variegation pattern and leaf color.

FIG. 4 shows an ‘LMV100’ leaf showing leaf size, variegation pattern, leaf colors and leaf apex shape.

FIG. 5 ‘LMV100’ foliage showing leaf attitude, variegation pattern and leaf colors.

### BOTANICAL DESCRIPTION OF THE PLANT

The following is a detailed botanical description of a new and distinct variety of a Lomandra longifolia ornamental grass-like plant known as ‘LMV100’. Plant observations were made on plants grown in New South Wales, Australia. Unless indicated otherwise, the descriptions disclosed herein are based upon observations made in May 2006 of mature ‘LMV100’ plants grown in nursery pots and field plots.

Those skilled in the art will appreciate that certain characteristics will vary with older or, conversely, younger plants. ‘LMV100’ has not been observed under all possible environmental conditions. Where dimensions, sizes, colors and other characteristics are given, it is to be understood that such characteristics are approximations or averages set forth as accurately as practicable. The phenotype of the variety may vary with variations in the environment such as season, temperature, light intensity, day length, cultural conditions and the like. Color notations are based on The Royal Horticultural Society Colour Chart, The Royal Horticultural Society, London, 1995 edition. ‘LMV100’ is undergoing further trialing and comparative testing in Australia and the United States.

‘LMV100’ is a perennial Lomandra longifolia plant which originated from a seedling selected from a population of open pollinated plants of Lomandra longifolia ‘Cassica’. After its selection, ‘LMV100’ was asexually propagated by division and subsequently by micropropagation. ‘LMV100’ has a variegated leaf blade combined with a medium density plant growth habit, which is unusual for Lomandra longifolia plants which usually have a non-variegated leaf blade.

Growth Habit, Dimensions and Color

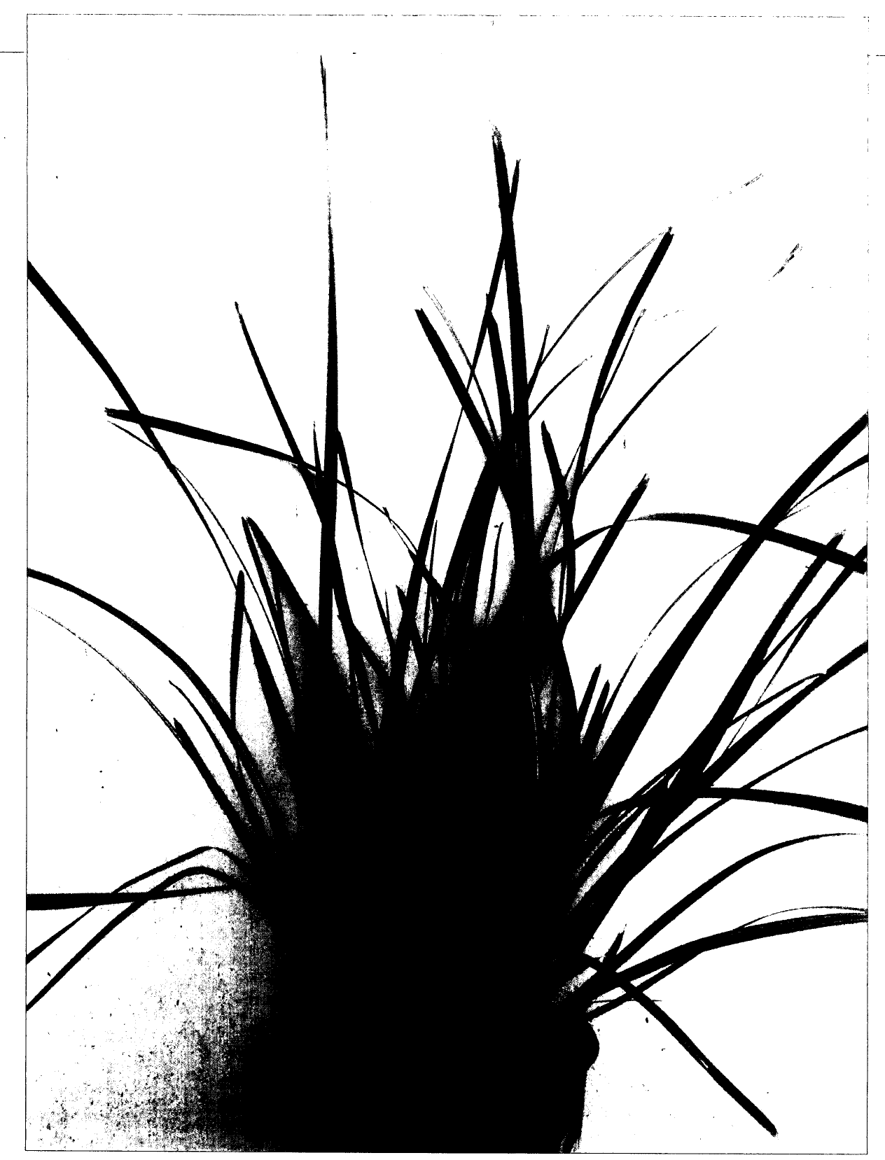

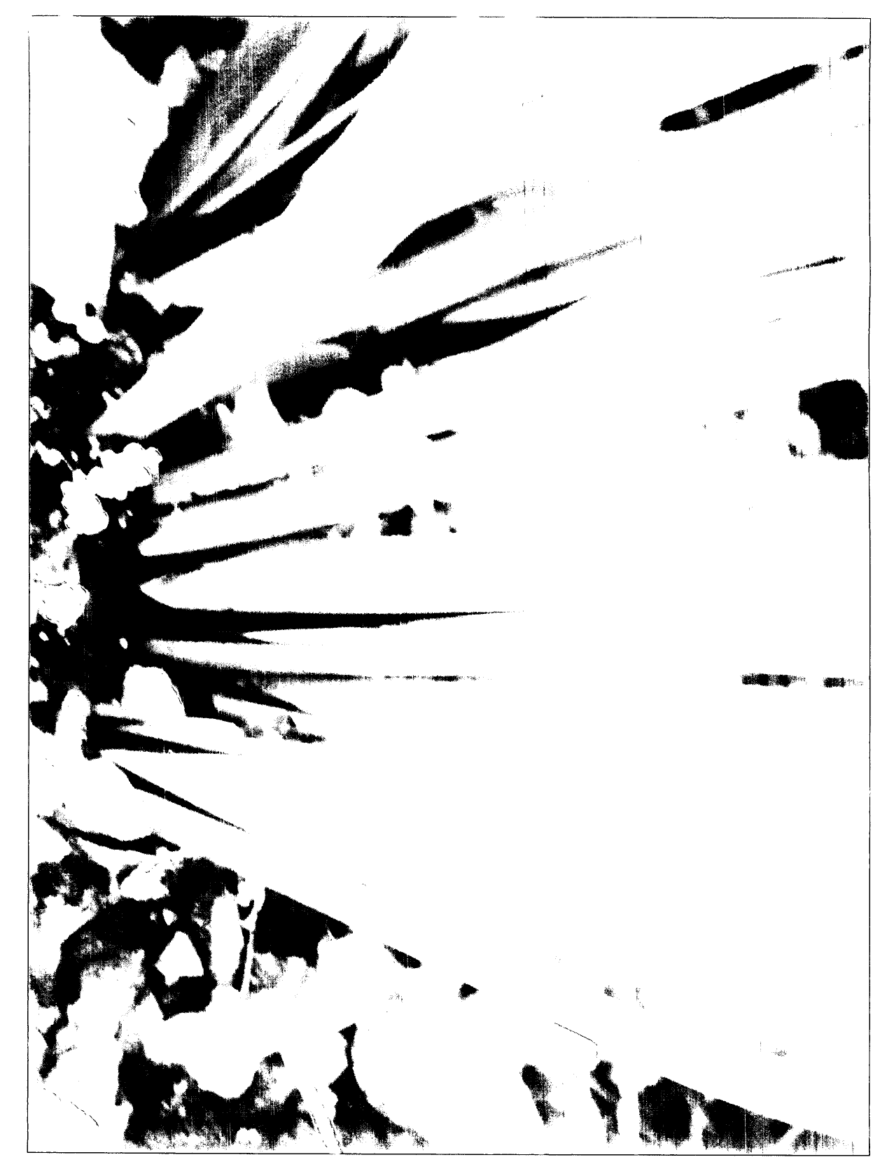

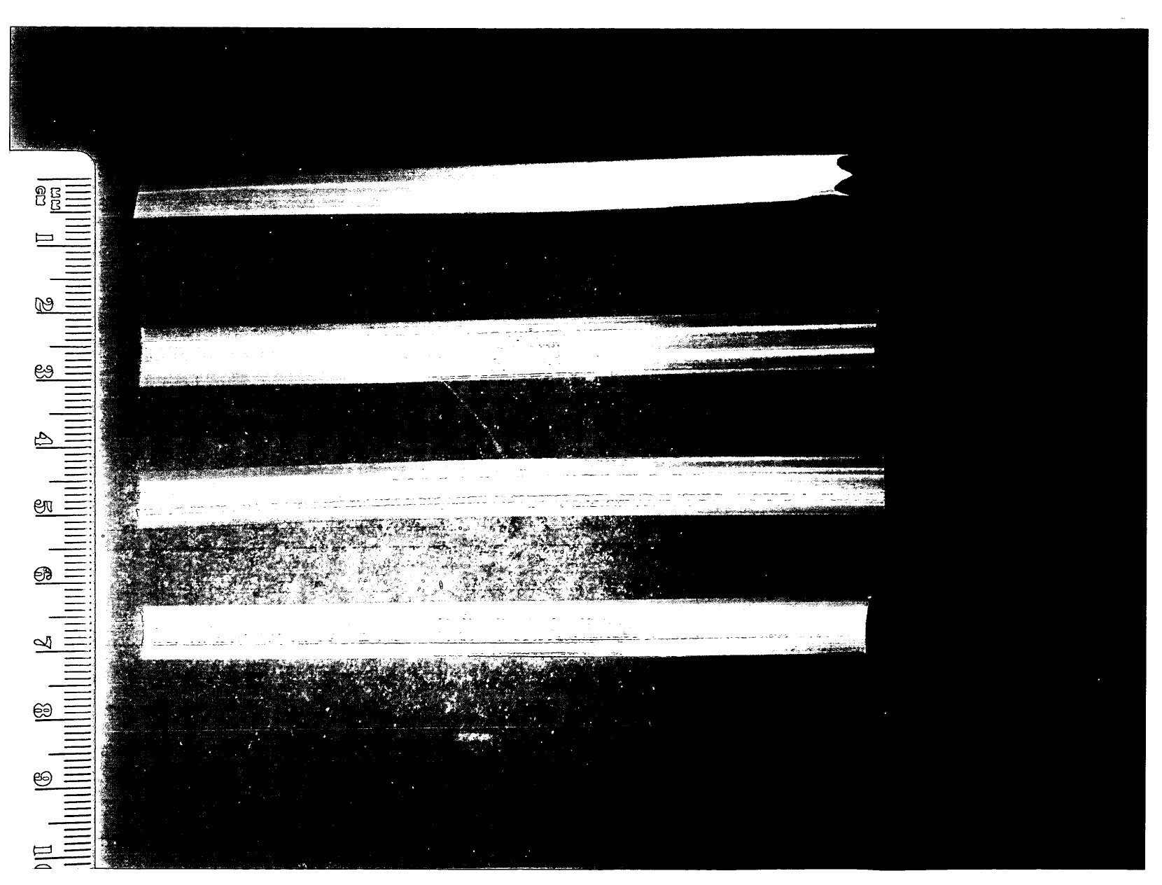

‘LMV100’ is a medium-tall, rhizomatous plant forming a upright to semi-upright tussock. Average plant height is 90 cm and average plant spread is 80 cm in a mature plant grown in a 300 mm nursery or field pot in Sydney, New South Wales, Australia. The upper and lower side of the leaf has a variegation consisting of striations of yellow green (circa RHS 146A-B) alternating with a yellow to yellow green color ranging from yellow green (circa RHS 154C) to yellow (circa RHS 5C). The leaf margin is typically colored with the yellow green with the variegation appearing as internal longitudinal bands of the yellow to yellow green color. The leaf blade is generally 6 to 10 mm in width, and up to 90 cm in length. The leaf base is cauline, leaf margin is entire, leaf apex is tridentate and leaf surface glaucosity is very weak to weak. The leaf venation pattern is parallel; the color is the same as the rest of the leaf. The basal part of shoots are a lighter color approximating yellow green (circa RHS 144B-C) in color variegated with green yellow (circa RHS 1C-D) with a basal leaf margin colored brown (circa RHS 200B) along the bottom 5-6 cm of leaf margin. The leaf is generally a uniform width from base to the tip, margins may shred at the base, with a tridentate apex typical of Lomandra longifolia. A representative ‘LMV100’ plant is shown in FIG. 1. A representative ‘LMV100’ plant base is shown in FIG. 2.
Roots: Similar to other Lomandra longifolia, ‘LMV100’ has a massive root structure. The roots are fibrous and spreading, similar to other Lomandra longifolia.
Rhizomes: Short (3 cm to 4 cm); rhizome color with the leaf sheath removed is white (RHS 155C); surface texture of the rhizome is smooth.
Inflorescence: The inflorescence is a spike (a panicle of clusters), composed of female flowers, typical of other Lomandra longifolia.
Seed: Flowers are female; seed has not produced so far. ‘LMV100’ has not been observed under all possible conditions or with all possible pollinators.
Flowering period: The primary flowering period is in the spring with secondary flowering occurring in summer to autumn in Sydney, New South Wales, Australia.
Lastingness of bloom: Approximately 9 days in summer flowering in Sydney, New South Wales, Australia.
Fragrance: Fragrance is typical of the species.
   Comparison of ‘LMV100’ with Other Varieties of Lomandra longifolia.—The leaf blade of ‘LMV100’ has a variegation present whereas variegation is absent with the varieties ‘Cassica’, ‘Katrinus’, ‘LM300’ (U.S. Plant Pat. No. 15,420) and ‘LM400’ (U.S. Plant Pat. No. 15,583) and the common form of Lomandra longifolia. The leaf blade variegation of ‘LMV100’ makes it an attractive ornamental grass-like plant.

‘LMV100’ has a medium leaf width whereas the varieties ‘LM300’ and ‘LM400’ have very narrow leaf widths. ‘LMV100’ has very weak to weak leaf glaucosity whereas ‘LM400’ has very strong leaf glaucosity The flower spike of ‘LMV100’ is similar to other Lomandra longifolia. The novelty and distinctiveness of ‘LMV100’ as compared with other varieties of Lomandra longifolia is still undergoing further testing.
Asexual Reproduction.—After its initial discovery, ‘LMV100’ was transplanted into a 140 mm pot for further trials and testing. After divisions were made for several subsequent generations, ‘LMV100’ was observed to retain plant growth characteristics that were noted in the original ‘LMV100’ seedling. ‘LMV100’ was then divided into many larger pots for further evaluation and introduction to micropropagation.
Environmental Tolerances.—‘LMV100’ has shown potential for shade tolerance and further shade tolerance tests are underway. The winter hardiness of ‘LMV100’ is at least to zone 8a in the Southeastern United States, and evaluation of winter hardiness is ongoing. ‘LMV100’ has been observed to hold color to −10 degrees Celsius without any noticeable change in appearance of the plant. ‘LMV100’ has excellent drought tolerance. After severe wilting, ‘LMV100’ has been noted to recover with watering. ‘LMV100’ does well in sandy soils, but also tolerates heavy, clay-type soils well.
Disease Resistance.—‘LMV100’ has good resistance to root rot compared with most other Lomandra longifolia and evaluation of disease resistance is ongoing. That which is claimed is:

1. A new and distinct variety of Lomandra longifolia plant named ‘LMV100’, substantially as described and illustrated herein.
